(19) United States
(12) Patent Application Publication
(10) Pub. No.: US 2006/0063832 A1
(43) Pub. Date: Mar. 23, 2006

(54) Pharmaceutical composition containing platinum complex as active substance and method of manufacturing thereof

(75) Inventors: Ales Franc, Brno (CZ); Peter Sova, Hradec Kralove (CZ)

Correspondence Address: NOTARO AND MICHALOS, 100 DUTCH HILL ROAD, SUITE 110, ORANGEBURG, NY, 10962-2100, US

(73) Assignee: PLIVA-LACHEMA A.S. A Czech Republic Corporation, 621 33 Brno (CZ)

(21) Appl. No.: 10/549,296

(22) PCT Filed: Mar. 30, 2004
(86) PCT No.: PCT/CZ04/00017

§ 371 Date: Sep. 15, 2005

(30) Foreign Application Priority Data
Mar. 31, 2003 (CZ) PV 2003-915
Feb. 12, 2004 (CZ) PV 2004-235

(51) Int. Cl. A61K 31/28; A61K 9/48; A61K 9/20

(52) U.S. Cl. 514/492; 424/451; 424/464

(57) ABSTRACT

The invention relates to a pharmaceutical composition containing the platinum complex of formula (I) as an active substance where A, A′, B, B′, X and X′ have specific meanings, in a mixture with at least one pharmaceutically acceptable excipient, characterized in that it is formed of a granulate with particles smaller than 0.5 mm in size prepared by wet granulation of a mixture of platinum complex of tetravalent platinum of formula (I) wetted by water, at least one neutral saccharide and at least one native and/or modified polysaccharide, being optionally contained in a capsule or a sack or being optionally pressed into the form of a tablet, while the surface of the granulate, the capsule or the tablet is optionally coated with a layer of at least one pharmaceutically acceptable substance enabling enterosolvent dissolution of the active substance in bowels only and/or at least one pharmaceutically acceptable substance enabling the controlled release of the active substance. The invention relates also the method of manufacturing of said pharmaceutical composition.

### TECHNICAL FIELD OF THE INVENTION

This invention relates to a solid pharmaceutical composition being usable for treating tumour diseases and containing tetravalent platinum complex as an active substance. This pharmaceutical composition ensures high stability of the active substance, and its enterosolvent and/or controlled release. This invention also relates to a method of manufacturing of the pharmaceutical composition mentioned.

### BACKGROUND OF THE INVENTION

It is generally known that platinum complexes have a broad anti-tumour effect that is used for treatment of many tumour diseases. Bivalent platinum complexes, particularly cisplatin, carboplatin or oxaliplatin have been used so far in the therapeutic praxis. These bivalent platinum complexes are unstable in the gastrointestinal tract and/or are absorbed only with difficulties. This fact makes the use of bivalent platinum complexes in an oral dosage form (that would be more suitable for patients) impossible. It was found out subsequently that some tetravalent platinum complexes are free of the drawback mentioned and keep their antitumour efficacy even if administered orally. These tetravalent platinum complexes were described as new chemical compounds for oral use in patent documents, namely RP 0 328 274, EP 0 423 707 and PCT/CZ99/00015.

The tetravalent platinum complexes, nevertheless, are generally almost insoluble in water (about 0.03 g/100 g), have small bulk density of about 0.2 g/ml, small tap density of about 0.4 g/ml and extremely high electrostatic charge. These physical properties represent a sigiificant problem for preparation of a solid pharmaceutical composition. In addition, tetravalent platinum complexes are chemically unstable when in contact with metals or many commonly used pharmaceutical excipients; this fact reduces the stability of the active substance in the pharmaceutical composition. The problems mentioned above have been partially successfully solved in the patent document PCT/CZ99/00015 where preparation of a solid pharmaceutical composition of the tetravalent platinum complex in the form of its soluble inclusion complexes with cyclodextrins, followed by its lyophilization is described. Nevertheless, this preparation is rather complex and costly. In addition, cyclodextrin capacity reduces significantly the content of platinum complex present in the inclusion complex mentioned above.

It is evident from the relevant prior art that the preparation of solid pharmaceutical compositions of tetravalent platinum complexes having good stability and sufficient content of the active substance has not been solved successfully yet.

### SUMMARY OF THE INVENTION

The invention mentioned provides the pharmaceutical composition containing the platinum complex of formula (I) as the active substance 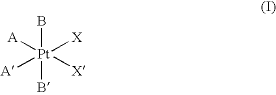
where

A and A′, independently of each other, are NH₃ group or the amine or diamine group containing 1 to 18 carbon atoms,

B and B′, independently of each other, are the halogen atom, the hydroxyl group or COOR or COOR′ group where R and R′, independently of each other, are hydrogen atom or alkyl, alkenyl, aryl, aralkyl, alkyl amine or alkoxyl group containing 1 to 10 carbon atoms or fimctional derivatives of the groups mentioned, and

X and X′, independently of each other, are halogen atom or the monocarboxylate group containing 1 to 20 carbon atoms, or

X and X′ together form the dicarbocylate group containing 2 to 20 carbon atoms, in a mixture with at least one pharmaceutically acceptable excipient characterized in that it is formed of a granulate with particles smaller than 0.5 mm in size prepared by wet granulation of a mixture of platinum complex of tetravalent platinum of formula (I) wetted by water, at least one neutral saccharide and at least one native and/or modified polysaccharide.

The pharmaceutical composition according to the invention is advantageously formed of the granulate prepared by wet granulation of the mixture of platinum complex of formula (I) wetted by water, at least one neutral sacchaiide at an amount equal to least 5% by weight and at least one native and/or modified polysaccharide at an amount equal to at least 2% by weight, related always to the total weight of the granulate.

The pharmaceutical composition according to the invention advantageously contains at least one pharmaceutically acceptable releasing agent and/or at least one pharmaceutically acceptable slipping substance.

The pharmaceutical composition according to the invention advantageously contains (OC-6-43)-bis(acetato)-(1-adamantylamine)-amine-dichloroplatinic complex as the active substance.

The mixture intended for wet granulation advantageously contains lactose, manritol, sorbitol, fRuctose, glucose and/or saccharose as the neutral saccharide.

The mixture intended for wet granulation advantageously contains maize, wheat and/or potato starch as the native and/or modified polysaccharide.

The pharmaceutical composition according to the invention is advantageously contained in a capsule or a sack or is pressed into a tablet form.

The surface of the granulate, capsule or tablet is advantageously coated with a layer of at least one pharmaceutically acceptable substance enabling enterosolvent dissolution of the active substance in bowels only, and/or with a layer of at least one pharmaceutically acceptable substance enabling controlled release of the active substance.

The surface of the granulate or the tablet is advantageously separated from the layer of at least one pharmaceutically acceptable substance enabling enterosolvent dissolution of the active substance in bowels only and/or from the layer of at least one pharmaceutically acceptable substance enabling the controlled release of the active substance by an inert closing layer consisting of at least one neutral saccharide, for example saccharose, and/or with at least one native and/or modified polysaccharide, for example native or modified maize, wheat or potato starch or gelatine or gum arabic, while the weight of the inert closing layer does not exceed 15% by weight, related to the total weight of the granulate or the tablet.

The layer of at least one pharmaceutically acceptable substance enabling the controlled release of the active substance is advantageously formed of ethyl cellulose and/or methacrylic acid and/or its compounds, advantageously polymers and/or copolymers of methacrylic acid, while the weight of the said layer is equal to not more than 40% by weight, related to the weight of the granulate, the capsule or the tablet.

The layer of at least one pharmaceutically acceptable substance enabling enterosolvent dissolution of the active substance in bowels only is advantageously formed of cellulose acetate and/or cellulose acetyl phthalate and/or cellulose acetosuccinate and/or hydroxypropylmethylcellulose phthalate and/or hydroxypropylmethylcellulose succinate and/or polyvinyl alcohol phthalate and/or benzophenyl salicylate and/or styrene copolymer with maleic acid and/or shellac and/or methacrylic acid and/or its compounds, advantageously polymers or copolymers of methacrylic acid while the weight of the said layer is equal to not more than 15% by weight, related to the weight of the granulate, the capsule or the tablet.

The invention relates also to a manufacturing method of the pharmaceutical composition according to the invention, characterized in that the mixture of platinum complex of formula (I) wetted by water, at least one neutral saccharide and at least one native and/or modified polysaccharide is granulated under wet conditions to obtain the granulate having particles smaller than 0.5 mm in size.

The wet granulation is advantageously performed to obtain granulate having such distribution of sizes of particles that 90% of them are smaller than 2.0 mm in size and not more than 20% of the particles are smaller than 0.09 mm in size.

The wet granulation is advantageously performed in an equipment, the surfaces of which, coming into contact with the granulated mixture, are inert to the said mixture.

The granulate is advantageously filled into capsules or sacks or, after at least one releasing agent and/or at least one slipping agent is added to the granulate, pressed into tablets.

The procedure of filling into capsules and sacks and of tablet-making is advantageously performed in the equipment, the surfaces of which, coming into contact with the mixture filled into capsules or sacks or with the mixture intended for tablet-making, are inert to the said riixture.

The granulate surface, the surface of the granulate to be filled into the sack, the tablet surface and the surface of the granulate to be filled into the capsule and/or the surface of the capsule mentioned are advantageously coated with a layer of at least one pharmaceutically acceptable substance enabling enterosolvent dissolution of the active substance in bowels only and/or a layer of at least one pharmaceutically acceptable substance enabling controlled release of the active substance.

The granulate surface, the surface of the granulate to be filled into the sack, the surface of the granulate to be filled into the capsule and the surface of the tablet, before being coated with the layer of at least one pharmaceutically acceptable substance enabling enterosolvent dissolution of the active substance in bowels only and/or the layer of at least one pharmaceutically acceptable substance enabling the controlled release of the active substance, are advantageously provided with an inert closing layer consisting of at least one neutral saccharide, for example saccharose, and/or at least one native and/or modified polysascharide, for example native or modified maize, wheat or potato starch or gelatine or gum arabic.

Coating of the granulate and the tablets with the inert closing layer, the layer of at least one pharmaceutically acceptable substance enabling enterosolvent dissolution of the active substance in bowels only or the layer of at least one pharmaceutically acceptable substance enabling the controlled release of the active substance is advantageously performed in equipment the surfaces of which, coming into contact with the granulate or the. tablets, are previously coated with the material forming the inert closing layer.

An example of the tetravalent platinum of formula (I) is (OC-6-43)-bis(acetato)-(1-adamantylamine)-amine-dichloroplatinic complex of formula (II) 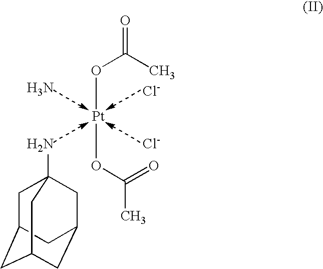
described in the Patent Application PCT/CZ99/00015.

It was found out within the invention framework that the platinum complex of formula (I) is unstable when being in contact with many mass-produced excipients, such as fillers, e.g. phosphates, sulfates or carbonates, standard slipping agents, binders and film-forming substances, e.g. esters of acrylic acid and their copolymers, cellulose derivatives of ethers, esters and copolymers series, or vinyl esters. Neutral saccharides used as fillers, native and/or modified polysaccharides used as binders, or possibly magnesium stearate used as the slipping agent, and native and/or modified polysaccharides used as the extragranulate releasing agents were found out to be the constitutional excipients with which the platinum complex of formula (I) is compatible and under their presence the complex mentioned is stable.

The resulting granulate, tablet or capsule is then advantageously coated with at least one layer of the film-forming substance ensuring enterosolvent and/or controlled release of the active substance. Due to the incompatibility of the active substance with many commonly used film-forming substances, the granulate and the tablets—before being coated with the said film-forming substance—are advantageously protected by a coating consisting of an inert closing layer that will protect the active substance from its decomposition and avoid migration of the film-forming substance into the granulate or tablet core. Neutral saccharide, e.g. saccharose, and/or the native and/or modified polysaccharide, e.g. native or modified maize, wheat or potato starch or gelatine or gum arabic, or possibly their mixtures in various ratios being in the form of the aqueous or aqueous-spirit hydrogel can be used as the material for the inert closing layer. This closing layer enables protection of the active substance from the enterosolvent coating and/or the coating enabling the controlled release of the active substance. The dry weight of the inert closing layer does not exceed 15% by weight, advantageously is 4-12% by weight, related to the total weight of the granulate or the tablet. The gelatine capsule does not need such protection because the material of the capsule itself protects effectively the active substance from the adverse effect of the film-forming substance.

In order to protect the active substance from the considerably acidic environment in stomach and/or in order to transfer the absorption spot of the active substance deeper into the gastrointestinal tract and thus to reach the increased value Tₘₐₓ, the granulate coated with the inert closing layer and the tablet coated with the inert closing layer or possibly the capsule are coated with an acid-resistant, i.e. enterosolvent, coating enabling the release of the active substance only in the small intestine, i.e. in the environment having pH value in the range of 4.5-8 according to the composition of the enterosolvent coating. The pharmaceutical composition prepared using this method complies with the requirements for testing of the enterosolvent dosage forms according to European Pharmacopoeia and/or US Pharmacopoeia, as amended. Cellulose acetate (CA), cellulose acetyl phthalate (CPA), cellulose acetosuccinate (CAS), hydroxypropylmethyl cellulose phthalate (MPMCP), hydroxypropylmethyl cellulose succinate (HPMCS), polyvinyl alcohol phthalate (PVAP), benzophenyl salicylate (BPS), styrene copolymer with maleic acid, shellac or copolymers of methacrylic acid, e.g. Eudragit L, Eudragit L-55 and Eudragit S, namely, both in the form of their plasticized aqueous dispersions—Eudragit L 30 D or L-55 30 D and Eudragid S 30 D—or in the form of the organic or aqueous-spirit solutions—Eudragit L 12.5 and Eudragit S 12.5—or possibly their mixtures in various ratios can be, for example, used as the film-forming substance, while the dry weight of the enterosolvent layer does not exceed 15% by weight, advantageously is 8-10% by weight, related to the weight of the granulate, the capsule or the tablet. The granulate coated with the inert closing layer or the tablets coated with the inert coating layer can be also filled directly into the capsules that are already treated for the enterosolvent application.

It was found out during experiments performed on dogs and pigs that a short-term high plasmatic concentration of the active substance occurs about one hour after the single application of the pharmaceutical composition, followed by its quick decrease. In order to reach more stable plasmatic levels of the active substance, enabling extension of the interval between individual applications of the pharmaceutical composition, and thus in order to reduce adverse effects resulting from the relatively high and shortly operating plasmatic levels of the active substance which occur after the application of the pharmaceutical composition with the instant release, the granulate protected by the inert closing layer or the tablets protected by the inert closing layer or the capsules can be, in addition, coated with a layer enabling controlled release of the active substance. The release of the active substance from the pharmaceutical composition treated as above complies with two limits, A and B, conforming to the amount of the released active substance per time specified by the dissolving test under the following conditions specified in the paddle method according to USP: Dissolving medium: 0.1 M of HCl; dissolving medium volume: 900 ml; paddle revolution speed: 100 rpm; dissolving medium temperature: 37° C. “A” limit in the given case is 5%-25% within 30 minutes, 15%-65% within 60 minutes, 40%-85% within 120 minutes and at least 85% within 180 minutes while “B” limit is 5%-25% within 60 minutes, 15%-65% within 180 minutes, 40%-85% within 360 minutes and at least 85% within 720 minutes. Ethyl cellulose (EC) or its aqueous dispersions—Surrelease or Aquacoat—or acrylate copolymers, e.g. Eudragit NE or Eudragit RL or Eudragit RS, both in the form of their plasticized aqueous dispersions—Eudragit NE 30 D, Eudragit RD 30 D, Eudragit RL 30 D—and in the form of their organic solutions—Eudragit RS 12.5 and Eudragit RL 12.5—or possibly their mixtures in various ratios can be, for example, used as the film-forming substance in case of the layer enabling the controlled release of the active substance, while the dry weight of the layer enabling the controlled release of the active substance does not exceed 40% by weight, advantageously is 8-30% by weight, related to the total weight of the granulate, the capsule or the tablet.

The granulate coated with the layer for controlled release of the active substance or the tablets coated with the layer for controlled release of the active substance can be also filled directly into the capsules being already treated for enterosolvent application or can be additionally coated with the enterosolvent layer. Surprisingly, it was found out during the preparation of the wet granulate of the pharmaceutical composition according to the invention that adverse chemical reactions occur on the surface of metals, which the pharmaceutical technological equipment intended for processing and manufacturing of solid pharmaceutical compositions is commonly produced from. This fact forestalls the use of standard manufacturing techniques, as, for example, compacting during granulate manufacturing or tablet-making, without a necessary surface treatment of dies. The wet granulate of the pharmaceutical composition according to the invention should be therefore advantageously processed in the equipment, the surfaces of which, coming into contact with the granulated mixture, are inert to the said mixture. Glass, porcelain, Teflon or enamel prove themselves as suitable inert materials.

When the granulate or the tablet are not coated with the inert closing layer, or when the granulate or the tablet are coated with the layer mentioned, and the inert closing layer intended for protection of the active substance in the granulate or the tablet from the effect of the materials of layers enabling the enterosolvent and/or controlled release of the active substance is damaged during the procedure when the granulate or the tablet is coated with the layers enabling the enterosolvent or controlled release, then the active substance comes into contact with metals if common coating equipment with metal surfaces is used, e.g. drum coating equipment, fluidization driers with upper feed, wusters or rotoprocessors. This can be, nevertheless, avoided so that the equipment surface, coming into contact with the pharmaceutical composition processed, is coated with the layer of the inert material that forms, at the same time, the material of the inert closing layer. When coating with the inert layer, the enterosolvent layer and/or the layer intended for the controlled release is performed in the identical equipment then the equipment surface mentioned can be coated already before the inert closing layer is applied.

The method used for preparation of the granulate forming the base of the pharmaceutical composition according to the invention, is wet granulation during which the mixture of platinum complex of formula (I) together with at least one neutral saccharide and at least one native and/or modified polysaccharide is wetted by water and mixed in a suitable mixer at a suitable speed and for a suitable time. The resulting granulate is then dried either under vacuum or under the atmospheric pressure. It was found out that the speed of the granulate dissolution is indirectly proportional to the size of the individual granules, and therefore the granulate is advantageously crushed in order to reach such distribution of sizes of particles that 90% of the particles are smaller than 2.0 mm and not more than 20% of the particles are smaller than 0.09 mm. Crushing, as mentioned above, is performed for example by milling in a ball mill or by manual or automated trituration in suitable devices.

The equipment intended for granulate filling into capsules or the tablet-making press intended for pressing granulate into tablets shall be inert to the granulate mentioned above in the contact surfaces, as has been already mentioned above.

The pharmaceutical composition according to the invention is characterized in that it has a good stability at the temperature of 40° C. and the relative humidity of 75%; this is supported by the fact that no relative increase in impurities exceeding 2% by weight was reported during 6 months and that the content of any individual unknown impurity does not exceed 0.1% by weight, related to the weight of the starting platinum complex of formula (II), after the time mentioned elapsed. No increase of the known impurity of platinum complex of formula (II), which is (acetato)-(1-adamantylamine)-amine-trichloro platinic complex having the formula [PtCl₃(ac) (am) (NH₃)].

The invention will be explained in more details in the examples of actual embodiments of the invention, while the examples mentioned are illustrative only and do not limit the scope of this invention that is unambiguously defined in the claims and the description.

### EXAMPLES

### Example 1

Composition and method of manufacturing of granulate having the pharmaceutical composition of platinum complex of formula (II)

Weights in the examples are given in parts by weight.

| | |
|---|---|
| 1. Platinum complex of formula (II) | 200.00 |
| 2. Modified maize starch | 20.00 |
| 3. Lactose, monohydrate | 200.00 |
| 4. Modified maize starch | 42.00 |
| 5. Magnesium stearate | 4.20 |

Procedure
Mix compounds Nos. 1 to 3 in a high-speed mixer.
Add 72-84 parts by weight of water.
Mix the mixture in the high-speed mixer for 2 minutes.
Dry granulate at the temperature of 70° C. until 2%-4% water content is obtained.
Mill dry granulate, e.g. in a jar mill, until 100% of the particles are smaller than 0.5 mm in size.
Add compounds Nos. 4 through 5 and mix in a cubic mixer for 15 minutes.

### Example 2

Method of filling of the granulate of the pharmaceutical composition of platinum complex of formula (II) prepared according to Example 1.
Fill the granulate obtained according to Example 1 and having the bulk and tap densities ranging between 0.4 g/ml and 0.6 g/ml and between 0.5 g/ml and 0.7 g/ml, respectively, manually or automatically into hard, normal or enterosolvent, gelatine or HPMC capsules sized 000. The weight of the filled granulate is 815.85 mg which corresponds to 350 mg of the active compound.

### Example 3

Method of filling of the granulate of the pharmaceutical composition of platinum complex of formula (II) prepared according to Example 1.
Fill the granulate obtained according to Example 1 and having the bulk and tap densities ranging between 0.4 g/ml and 0.6 g/ml and between 0.5 g/ml and 0.7 g/ml, respectively, manually or automatically into hard, normal or enterosolvent, gelatine or HPMC capsules sized 00 or 000 or press it into tablets. The weight of the filled granulate is 582.75 mg which is 250 mg of the active compound.

### Example 4

Method of filling of the granulate of the pharmaceutical composition of platinum complex of formula (II) prepared according to Example 1.
Fill the granulate obtained according to Example 1 and having the bulk and tap densities ranging between 0.4 g/ml and 0.6 g/ml and between 0.5 g/ml and 0.7 g/ml, respectively, manually or automatically into hard, normal or enterosolvent, gelatine or HPMC capsules sized 00 or 0 or press it into tablets. The weight of the filled granulate is 466.20 mg which is 200 mg of the active compound.

### Example 5

Method of filling of the granulate of the pharmaceutical composition of platinum complex of formula (II) prepared according to Example 1.
Fill the granulate obtained according to Example 1 and having the bulk and tap densities ranging between 0.4 g/ml and 0.6 g/ml and between 0.5 g/ml and 0.7 g/ml, respectively, manually or automatically into hard, normal or enterosolvent, gelatine or HPMC capsules sized 0 or 1 or press it into tablets. The weight of the filled granulate is 349.65 mg which is 150 mg of the active compound.

### Example 6

Method of filling of the granulate of the pharmaceutical composition of platinum complex of formula (II) prepared according to Example 1.
Fill the granulate obtained according to Example 1 and having the bulk and tap densities ranging between 0.4 g/ml and 0.6 g/ml and between 0.5 g/ml and 0.7 g/ml, respectively, manually or automatically into hard, normal or enterosolvent, gelatine or HPMC capsules sized 1 or 2 or press it into tablets. The weight of the filled granulate is 233.10 mg which is 100 mg of the active compound.

### Example 7

Method of filling of the granulate of the pharmaceutical composition of platinum complex of formula (II) prepared according to Example 1
Fill the granulate obtained according to Example 1 and having the bulk and tap densities ranging s between 0.4 g/ml and 0.6 g/ml and between 0.5 g/ml and 0.7 g/ml, respectively, manually or automatically into hard, normal or enterosolvent, gelatine or HPMC capsules sized 2 or 3 or press it into tablets. The weight of the filled granulate is 174.825 mg which is 75 mg of the active compound.

### Example 8

Method of filling of the granulate of the pharmaceutical composition of platinum complex of formula (II) prepared according to Example 1.
Fill the granulate obtained according to Example 1 and having the bulk and tap densities ranging between 0.4 g/ml and 0.6 g/ml and between 0.5 g/ml and 0.7 g/ml, respectively, manually or automatically into hard, normal or enterosolvent, gelatine or HPMC capsules sized 3 or 4 or press it into tablets. The weight of the filled granulate is 116.55 mg which is 50 mg of the active compound.

### Example 9

Composition and method of manufacturing of the granulate having the pharmaceutical composition of platinum complex of formula (II)

Weights in the examples are given in parts by weight

| | |
|---|---|
| 1. Platinum complex of formula (II) | 200.00 |
| 2. Modified maize starch | 62.00 |
| 3. Lactose, monohydrate | 200.00 |
| 4. Modified maize starch | 4.20 |

Procedure
Mix compounds Nos. 1 to 3 in a high-speed mixer.
Add 80-120 parts by weight of water.
Mix the mixture in the high-speed mixer for 2 minutes.
Dry granulate at the temperature of 70° C. until 2%-4% of water content is obtained.
Mill dry granulate, e.g. in a jar mill, until 90% of the particles are smaller than 2.0 mm in size and not more than 20% of them are smaller than 0.09 mm in size.
Add compound No. 4 and mix in a cubic mixer for 15 minutes.

### Example 10

Method of filling of the granulate of the pharnaceutical composition of platinum complex of formula (II) prepared according to Example 9.
Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized 000 or 00. The weight of the filled granulate is 815.85 mg which corresponds to 350 mg of the active compound.

### Example 11

Method of filling of the granulate of the pharmaceutical composition of platinum complex of formula (II) prepared according to Example 9.
Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized between 000 and 0 or press it into tablets. The weight of the filled granulate is 582.75 mg which is 250 mg of the active compound.

### Example 12

Method of filling of the granulate of the pharmaceutical composition of platinum complex of formula (II) prepared according to Example 9.
Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized 00 or 0 or press it into tablets. The weight of the filled granulate is 466.20 mg which is 200 mg of the active compound.

### Example 13

Method of filling of the granulate of the pharmaceutical composition of platinum complex of formula (II) prepared according to Example 9.
Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized between 0 and 2 or press it into tablets. The weight of the filled granulate is 349.65 mg which is 150 mg of the active compound.

### Example 14

Method of filling of the granulate of the pharmaceutical composition of platinum complex of formula (II) prepared according to Example 9.
Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized between 1 and 3 or press it into tablets. The weight of the filled granulate is 233.10 mg which corresponds to 100 mg of the active compound.

### Example 15

Method of filling of the granulate of the pharmaceutical composition of platinum complex of formula (II) prepared according to Example 9.

Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized between 2 and 4 or press it into tablets. The weight of the filled granulate is 174.825 mg which corresponds to 75 mg of the active compound.

### Example 16

Method of filling of the granulate of the pharmaceutical composition of platinum complex of formula (II) prepared according to Example 9.
Fill the granulate obtained according to Example 9 and having the bulk and tap densities ranging between 0.4 g/ml and 0.7 g/ml and between 0.5 g/ml and 0.8 g/ml, respectively, manually or automatically into hard, normal or enterosolvent, gelatine or HPMC capsules sized between 3 and 5 or press it into tablets. The weight of the filled granulate is 116.55 mg which is 50 mg of the active compound.

### Example 17

Method of the application of the closing layer onto the granulate of the pharmaceutical composition of platinum complex of formula (II) prepared according to Example 9 in a fluid bed.

| | |
|---|---|
| Equipment: | Wurster |
| Granulate charge: | 0.50 kg |
| Temperature of inlet air: | 50° C.-70° C. |
| Temperature of outlet air: | 30° C.-50° C. |
| Injection speed: | 6-25 g/minute |
| Nozzle diameter: | 0.8 mm |
| Weight of coating layer: | 4-20% by weight |

Perform the injection until the target granulate weight, corresponding to the demanded weight of the coating layer, is reached.

Use 64% by weight of the saccharose solution or 8% by weight of starch hydrogel prepared from modified maize starch dissolved under cold conditions or from native maize starch dissolved at the temperature of 70° C. Alternatively, use aqueous hydrogel of a mixture of 4% by weight of gum arabic and 5% by weight of gelatine A or B.

The standard drum coating equipment can be also used for the preparation as above.

### Example 18

Method of the application of the closing layer onto tablets of the pharmaceutical composition of platinum complex of formula (II) prepared according to Examples 2 to 16 in a fluid bed.

| | |
|---|---|
| Equipment: | Wurster |
| Core charge: | 0.50 kg |
| Temperature of inlet air: | 50° C.-70° C. |
| Temperature of outlet air: | 40° C.-60° C. |
| Injection speed: | 6-18 g/minute |
| Nozzle diameter: | 0.8 mm |
| Weight of coating layer: | 4-8% by weight |

Perform the injection until the target granulate weight, corresponding to the demanded weight of the coating layer, is reached.

Use 64% by weight of saccharose solution or 8% by weight of starch hydrogel prepared from modified maize starch dissolved under cold conditions or from native maize starch dissolved at the temperature of 70° C. Alternatively, use aqueous hydrogel of a mixture of 4% by weight of gum arabic and 5% by weight of gelatine A or B.

The standard drum coating equipment can be also used for the preparation as above.

### Example 19

Method of the application of the enterosolvent layer onto the granulate of the pharmaceutical composition of platinum complex of formula (II) prepared according to Example 17 in a fluid bed.

| | |
|---|---|
| Equipment: | Wurster |
| Granulate charge: | 0.50 kg |
| Temperature of inlet air: | 50° C.-70° C. |
| Temperature of outlet air: | 24° C.-50° C. |
| Injection speed: | 6-25 g/minute |
| Nozzle diameter: | 0.8 mm |
| Weight of coating layer: | 8-12% by weight |

Perform the injection until the target granulate weight, corresponding to the demanded weight of the coating layer, is reached.

Use 20% by weight of Eudradit L aqueous dispersion or 10% by weight of HPMCP aqueous dispersion.

The standard drum coating equipment can be also used for the preparation as above.

### Example 20

Method of the application of the enterosolvent layer onto tablets and capsules of the pharmaceutical composition of platinum complex of formula (II) prepared according to Example 18 (tablets) and according to Examples 2 to 16 (capsules) in a fluid bed.

| | |
|---|---|
| Equipment: | Wurster |
| Core charge: | 0.50 kg |
| Temperature of inlet air: | 50° C.-70° C. |
| Temperature of outlet air: | 25° C.-60° C. |
| Injection speed: | 6-18 g/minute |
| Nozzle diameter: | 0.8 mm |
| Weight of coating layer: | 8-10% by weight |

Perform the injection until the target granulate weight, corresponding to the demanded weight of the coating layer, is reached.

Use 20% by weight of Eudradit L aqueous dispersion or 10% by weight of HPMCP aqueous dispersion.

The standard drum coating equipment can be also used for the preparation as above.

### Example 21

Method of the application of the layer intended for the controlled release onto the granulate of the pharmaceutical composition of platinum complex of formula (II) prepared according to Example 17 in a fluid bed.

| | |
|---|---|
| Equipment: | Wurster |
| Granulate charge: | 0.50 kg |
| Temperature of inlet air: | 50° C.-70° C. |
| Temperature of outlet air: | 24° C.-50° C. |
| Injection speed: | 6-25 g/minute |
| Nozzle diameter: | 0.8 mm |
| Weight of coating layer: | 10-30% by weight |

Perform the injection until the target granulate weight, corresponding to the demanded weight of the coating layer, is reached.

Use 15% by weight of Ethyl cellulose aqueous dispersion (Surrelease) for the application. Alternatively, 20% (weight/weight) plasticized Eudragit RS or RP lacquer dispersion or their mixture in a suitable ratio can be used.

The standard drum coating equipment can be also used for the preparation as above.

### Example 22

Method of the application of the layer intended for the controlled release onto the tablets and the capsules of the pharmaceutical composition of platinum complex of formula (II) prepared according to Example 18 (tablets) and according to Examples 2 to 16 (capsules) in a fluid bed.

| | |
|---|---|
| Equipment: | Wurster |
| Core charge: | 0.50 kg |
| Temperature of inlet air: | 50° C.-70° C. |
| Temperature of outlet air: | 24° C.-50° C. |
| Injection speed: | 4-18 g/minute |
| Nozzle diameter: | 0.8 mm |
| Weight of coating layer: | 8-20% by weight |

Perform the injection until the target granulate weight, corresponding to the demanded weight of the coating layer, is reached.

Use 15% by weight of Ethyl cellulose aqueous dispersion (Surrelease) for the application. Alternatively 20% (weight/weight) plasticized Eudragit RS or RP aqueous dispersion or their mixture in a suitable ratio can be used.

The standard drum coating equipment can be also used for the preparation as above.

### Example 23

The granulates and the tablets prepared according to Examples 21 and 22 can be filled into hard gelatine capsules treated for the release in bowels.

### Example 24

The granulates coated with the layer intended for the controlled release prepared according to Example 21 can be, in addition, coated with the layer intended for enterosolvent release according to Example 19.

### Example 25

The tablets coated with the layer intended for the controlled release, prepared according to Example 22 can be, in addition, coated with the layer intended for enterosolvent release according to Example 20.

### Example 26

Stability testing of the granulate of the pharmaceutical composition of platinum complex of formula (II) prepared according to Example 1.

The capsules prepared using the procedure according to Example 4 and Example 8, filled into HDPE containers that were stored at the temperature of 40° C. and relative humidity of 75% for 6 months were used for stability testing. The sum of the unknown impurities did not exceed 2% by weight during the period mentioned and no individual unknown impurity exceeded 0.1% by weight, related to the starting platinum complex of formula (II).

### Example 27

The time course of the release of the active substance from the pharmaceutical composition having the form of hard gelatine capsules, prepared according to Example 4 and Example 8

The conditions of the dissolution test—according to USP, paddle method

| | |
|---|---|
| Medium: | 0.1 M of HCl, 900 ml |
| Speed: | 100 rpm |
| Medium temperature: | 37° C. |

The amount of the active substance released is given in % by weight

| | % of released substance | % of released substance |
|---|---|---|
| | Contents of capsules | Contents of capsules |
| Time (minutes) | containing 50 mg | containing 200 mg |
| 2 | 0.7 | 0.3 |
| 4 | 12.6 | 4.9 |
| 6 | 44.6 | 24.5 |
| 8 | 57.6 | 44.7 |
| 10 | 65.0 | 54.3 |
| 12 | 69.9 | 60.5 |
| 15 | 74.9 | 66.0 |
| 20 | 80.4 | 72.3 |
| 30 | 86.6 | 79.5 |
| 40 | 90.5 | 84.3 |
| 50 | 93.1 | 87.4 |
| 60 | 95.1 | 89.5 |

### Example 28

Tome limits, A and B, for release of the active substance from the pharmaceutical composition having the form of the granulates, the hard gelatine capsules and the tablets prepared according to Examples 21 and 22 intended for the controlled release of the medicinal product

The conditions of the dissolution test—according to USP, paddle method

| | |
|---|---|
| Medium: | 0.1 M of HCl, 900 ml |
| Speed: | 100 rpm |
| Medium temperature: | 37° C. |

| | Limit “A”: | Limit “B”: |
|---|---|---|
| | % of released medicinal | % of released medicinal |
| Time (minutes) | product | product |
| 30 | 5-25 | — |
| 60 | 15-65 | 5-25 |
| 120 | 40-85 | — |
| 180 | At least 85 | 15-65 |
| 240 | — | — |
| 360 | — | 40-85 |
| 720 | — | At least 85 |

1. A pharmaceutical composition containing platinum complex of formula (I) as an active substance 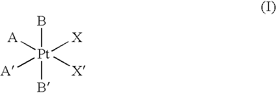
where
A and A′, independently of each other, are NH₃ group or amine or diamine group containing 1 to 18 carbon atoms,
B and B′, independently of each other, are halogen atom, hydroxyl group or COOR or COOR′ group where R and R′, independently of each other, are hydrogen atom or alkyl, alkenyl, aryl, aralkyl, alkyl amine or alkoxyl group containing 1 to 10 carbon atoms or functional derivatives of the said groups, and
X and X′, independently of each other, are halogen atom or monocarboxylate group containing 1 to 20 carbon atoms, or
X and X′ together form dicarboxylate group containing 2 to 20 carbon atoms, in a mixture with at least one pharmaceutically acceptable excipient characterized in that it is formed of a granulate with particles smaller than 0.5 mm in size prepared by wet granulation of a mixture of platinum complex of tetravalent platinum of formula (I) wetted by water, at least one neutral saccharide and at least one native and/or modified polysaccharide.

2. The pharmaceutical composition according to claim 1 characterized in that it is formed of the granulate prepared by wet granulation of the mixture of platinum complex of formula (I), at least one neutral saccharide at an amount equal to at least 5% by weight and at least one native and/or modified polysaccharide at an amount equal to at least 2% by weight, related always to the total weight of the granulate.

3. The pharmaceutical composition according to claim 1 characterized in that it contains at least one pharmaceutically acceptable releasing agent and/or at least one pharmaceutically acceptable slipping substance.

4. The pharmaceutical composition according to claim 1 characterized in that it contains (OC-6-43)-bis(acetato)-(1-adamantylamine)-amine-dichloroplatinic complex as the active substance.

5. The pharmaceutical composition according to claim 1 characterized in that the mixture intended for wet granulation contains lactose, mannitol, sorbitol, fructose, glucose and/or saccharose as the neutral saccharide.

6. The pharmaceutical composition according to claim 1 characterized in that the mixture intended for wet granulation contains maize, wheat and/or potato starch as the native and/or modified polysaccharide.

7. The pharmaceutical composition according to claim 1 characterized in that it is contained in a capsule or a sack or is pressed into a tablet form.

8. The pharmaceutical composition according to claim 1 characterized in that the surface of the granulate, the capsule or the tablet is coated with a layer of at least one pharmaceutically acceptable substance enabling enterosolvent dissolution of the active substance in bowels only, and/or with a layer of at least one pharmaceutically acceptable substance enabling controlled release of the active substance.

9. The pharmaceutical composition according to claim 8 characterized in that the surface of the granulate or the tablet is separated from the layer of at least one pharmaceutically acceptable substance enabling enterosolvent dissolution of the active substance in bowels only and/or from the layer of at least one pharmaceutically acceptable substance enabling the controlled release of the active substance with an inert closing layer consisting of at least one neutral saccharide, for example saccharose, and/or with at least one native and/or modified polysaccharide, for example native or modified maize, wheat or potato starch or gelatine or gum arabic, while the weight of the inert closing layer does not exceed 15% by weight, related to the total weight of the granulate or the tablet.

10. The pharmaceutical composition according to claim 8 characterized in that the layer of at least one pharmaceutically acceptable substance enabling the controlled release of the active substance is formed of ethyl cellulose and/or methacrylic acid and/or its compounds, advantageously polymers and/or copolymers of methacrylic acid, while the weight of the said layer is equal to not more than 40% by weight, related to the weight of the granulate, the capsule or the tablet.

11. The pharmaceutical composition according to claim 8 characterized in that the layer of at least one pharmaceutically acceptable substance enabling enterosolvent dissolution of the active substance in bowels only is formed of cellulose acetate and/or cellulose acetyl phthalate and/or cellulose acetosuccinate and/or hydroxypropylmethylcellulose phthalate and/or hydroxypropylmethylcellulose succinate and/or polyvinyl alcohol phthalate and/or benzophenyl salicylate and/or styrene copolymer with maleic acid and/or shellac and/or methacrylic acid and/or its compounds, advantageously polymers or copolymers of methacrylic acid while the weight of the said layer is equal to not more than 15% by weight, related to the weight of the granulate, the capsule or the tablet.

12. A method of manufacturing of the pharmaceutical composition according to claim 1 characterized in that the mixture of platinum complex of formula (I) wetted by water, at least one neutral saccharide and at least one native and/or modified polysaccharide is granulated under wet conditions to obtain granulate consisting of particles smaller than 0.5 mm in size.

13. The method according to claim 12 characterized in that the wet granulation is performed to obtain the granulate having such distribution of sizes of particles that 90% of them are smaller than 2.0 mm in size and not more than 20% of the particles are smaller than 0.09 mm in size.

14. The method according to claim 13 characterized in that the wet granulation is performed in equipment, the surfaces of which, coming into contact with the granulated mixture are inert to said mixture.

15. The method according to claim 12 characterized in that the granulate is filled into a capsule or a sack or, after at least one releasing agent and/or at least one slipping agent is added to the granulate, pressed into tablets.

16. The method according to claim 15 characterized in that filling into capsules and sacks and tablet-making is performed in equipment, the surfaces of which, coming into contact with the mixture filled into capsules or sacks or with the mixture intended for tablet-making are inert to said mixture.

17 The method according to claim 12 characterized in that the granulate surface, the surface of the granulate to be filled into the sack, the tablet surface and the surface of the granulate to be filled into the capsule and/or the surface of the capsule mentioned are coated with a layer of at least one pharmaceutically acceptable substance enabling enterosolvent dissolution of the active substance in bowels only and/or a layer of at least one pharmaceutically acceptable substance enabling the controlled release of the active substance.

18 The method according to claim 17 characterized in that the granulate surface, the surface of the granulate to be filled into the sack, the surface of the granulate to be filled into the capsule and the surface of a tablet, before being coated with the layer of at least one pharmaceutically acceptable substance enabling enterosolvent dissolution of the active substance in bowels only and/or the layer of at least one pharmaceutically acceptable substance enabling the controlled release of the active substance, are provided with an inert closing layer formed of at least one neutral saccharide, for example saccharose, and/or at least one native and/or modified polysaccharide, for example native or modified maize, wheat or potato starch or gelatine or gum arabic.

19 The method according to claim 18 characterized in that coating of the granulate and the tablets with the inert closing layer, the layer of at least one pharmaceutically acceptable substance enabling enterosolvent dissolution of the active substance in bowels only or the layer of at least one pharmaceutically acceptable substance enabling the controlled release of the active substance is performed in equipment, the surfaces of which, coming into contact with the granulate or the tablets are previously coated with a material forming the inert closing layer.
